(19) United States
(12) Patent Application Publication
(10) Pub. No.: US 2016/0213207 A1
(43) Pub. Date: Jul. 28, 2016

(54) URINAL DEVICE

(71) Applicant: Jay W. KIM, La Mirada, CA (US)

(72) Inventor: Jay W. KIM, La Mirada, CA (US)

(21) Appl. No.: 14/992,409

(22) Filed: Jan. 11, 2016

Related U.S. Application Data
(60) Provisional application No. 62/106,376, filed on Jan. 22, 2015.

(51) Int. Cl. A47K 11/12; A61F 5/453

(52) U.S. Cl. CPC A47K 11/12; A61F 5/453

(57) ABSTRACT

A urinal device comprises an upper opening, a reservoir portion, a funnel portion, a drain pipe portion, and a lower opening. The upper opening is configured for receiving urine from above. The reservoir portion extends downwards from and is formed below the upper opening and configured for holding urine received through the upper opening temporarily. The funnel portion extends downwards from and is formed below the reservoir portion and configured for draining urine from the reservoir portion. The drain pipe portion extends downwards from and is formed below the funnel portion and configured for draining urine from the funnel portion. The lower opening is formed at a lowest end of the drain pipe portion. The reservoir portion has a largest cross-sectional area between the upper opening and the funnel portion, so that the reservoir portion has a volume enough to hold urine.

### RELATED APPLICATION

This application is a Non-provisional Application of Provisional Application Ser. No. 62/106,376 for “Urinal Device” filed on Jan. 22, 2015.

### BACKGROUND OF THE INVENTION

The present invention relates to a urinal device.

For men, the conventional urinals are not inconvenient to use, but they are not everywhere. Usually, the urinals are provided in public buildings, but not in single family houses.

According to recent researches, when the urine falls into the closet bowl, which is the only available in the regular houses, micro-drops of urine fly away from the bowl due to the impact against water surface in the bowl. Even though they are invisible to naked eyes, sometimes the micro-drops of urine are sputtered and fly almost everywhere, for example, even up to the ceiling.

Therefore, the micro-drops of urine reach nearly everywhere in the bathroom, contaminating invisibly. In a word, it is a situation of insanitary conditions.

Accordingly, a need for a urinal device for men has been present for a long time considering the expansive demands in the everyday life. This invention is directed to solve these problems and satisfy the long-felt need.

### SUMMARY OF THE INVENTION

The present invention contrives to solve the disadvantages of the prior art.

An object of the invention is to provide a urinal device for men.

The urinal device for men is portable, so that the user holds the urinal device with a single hand, and pees into the urinal device.

The urinal device is configured to receive urine and guide them down into the closet bowl safely. Since the free-fall of urine is mitigated by the inner walls of the urinal, the impact to the bowl water surface are reduced much, so as to land on the water surface without splashes.

In certain embodiments of the invention, the urinal device may comprise a reservoir portion provided in the middle thereof, which provides a reservoir having more volume for holding flood of urine temporarily until it takes time to be drained downwards.

Additionally, the urinal device may comprise a handle, which the user can hold. The shape and size of the handle may be determined by a design choice and environments and conditions of the user.

A urinal device comprises an upper opening, a reservoir portion, a funnel portion, a drain pipe portion, and a lower opening.

The upper opening is configured for receiving urine from above.

The reservoir portion extends downwards from and is formed below the upper opening and configured for holding urine received through the upper opening temporarily.

The funnel portion extends downwards from and is formed below the reservoir portion and configured for draining urine from the reservoir portion.

The drain pipe portion extends downwards from and is formed below the funnel portion and configured for draining urine from the funnel portion.

The lower opening is formed at a lowest end of the drain pipe portion.

The reservoir portion has a largest cross-sectional area between the upper opening and the funnel portion, so that the reservoir portion has a volume enough to hold urine.

A portion connecting from the upper opening to the reservoir portion may be tilted like a portion of a reversed funnel, as in a middle-bulged wine cup.

The urinal device may further comprise a rubber pad along the upper opening.

The urinal device may further comprise a handle extending from an edge of the upper opening or the reservoir portion.

The upper opening may be disposed flatly or obliquely with respect to a horizontal direction.

The reservoir portion may be bulged to a single direction only.

The urinal device may further comprise a stand having a stable bottom portion and a receiving hole provided on a top portion, which is configured to receive the lower part of the drain pipe portion and the lower opening.

The stable bottom portion may be heavy enough to provide a center-of-mass low enough for the urinal device to stand stably.

The top portion of the stand may extend upwards from the stable bottom portion.

The urinal device may further comprise an extension pipe, to which the lower part of the drain pipe portion close to the lower opening is configured to be inserted.

The extension pipe is detachable.

The reservoir portion may be made of flexible material.

The urinal device may be made of plastic integrally.

The advantages of the present invention are: (1) the urinal device for men according to the invention is easy to make; and (2) the urinal device for men according to the invention is convenient to use.

Although the present invention is briefly summarized, the fuller understanding of the invention can be obtained by the following drawings, detailed description and appended claims.

### BRIEF DESCRIPTION OF THE DRAWINGS

These and other features, aspects and advantages of the present invention will become better understood with reference to the accompanying drawings, wherein:

FIG. 1 shows a urinal device according to an embodiment of the invention;

FIG. 2 shows a prior art;

FIG. 3 shows a urinal device according to another embodiment of the invention;

FIG. 4 shows a prior art;

FIG. 5 shows a urinal device according to still another embodiment of the invention;

FIG. 6 shows a urinal device according to another embodiment of the invention;

FIG. 7 shows a urinal device according to another embodiment of the invention;

FIG. 8 shows a urinal device according to another embodiment of the invention;

FIGS. 9-12 show different views of a urinal device according to another embodiment of the invention;

FIGS. 13-15 show different views of a urinal device according to another embodiment of the invention;

FIGS. 16-17 show different views of a urinal device with a stand according to still another embodiment of the invention;

FIGS. 18-21 show different views of a urinal device with a stand according to still another embodiment of the invention;

FIGS. 22-23 show different views of a stand for a urinal device according to still another embodiment of the invention;

FIGS. 24 and 25 show different views of a urinal device according to still another embodiment of the invention;

FIG. 26 shows a user using a urinal according to a prior art; and

FIG. 27 shows a user using a urinal according to embodiments of the invention.

### DETAILED DESCRIPTION EMBODIMENTS OF THE INVENTION

The U.S. Provisional Application Ser. No. 62/106,376 for “Urinal Device” filed on Jan. 22, 2015 is incorporated by reference herein for any and all purposes.

Referring to the figures, the embodiments of the invention are described in detail.

An object of the invention is to provide a urinal device for men, which helps a user to be more sanitary in the restroom.

The urinal device (100) comprises an upper opening (10), a reservoir portion (20), a funnel portion (30), a drain pipe portion (40), and a lower opening (50).

The upper opening (10) is configured to receive urine. The size and shape of the upper opening (10) are determined to facilitate the receiving of urine. Therefore, the illustrated sizes and shapes of the upper opening (10) are just for exemplary ones, not limiting.

The reservoir portion (20) extends downwards from the upper opening (10) and has a lager cross-sectional dimension than the upper opening (10). In a case of circular cross-section, the diameter of the reservoir portion (20) is larger than the diameter of the upper opening (10), so as to provide larger volume to hold urine for an extended time period until the urine is drained down. The size and shape of the reservoir portion (20) are again determined by design choices. So, the illustrated sizes and shapes of the reservoir portion (20) are just for exemplary ones, not limiting.

Also, the portion connecting from the upper opening (10) to the reservoir portion (20) may be tilted like a portion of reversed funnel, as in a middle-bulged wine cup.

The funnel portion (30) extends downwards from the reservoir portion (20) with cross-sectional dimension (or diameter as illustrated) decreasing to a specific size.

The drain pipe portion (40) extends downwards from the funnel portion (30) and configured to drain urine from above to the lower opening (50).

In usage, the lower opening (50) is aimed into the closet bowl.

The urinal device (100) may be made of plastic material. Of course, it is not limited to that.

The urinal device for men is portable, so that the user holds the urinal device with a single hand, and pees into the urinal device (100).

The urinal device (100) is configured to receive urine and guide them down into the toilet bowl safely. Since the free-fall of urine is mitigated by the inner walls of the urinal device(100), the impact to the bowl water surface are reduced much, so as to land on the water surface without splashes.

In certain embodiments of the invention, the urinal device (100) may comprise a reservoir portion (20) provided in the middle thereof, which provides a reservoir having more volume for holding flood of urine temporarily until it takes time to be drained downwards.

Additionally, the urinal device (100) may comprise a handle (60), which the user can hold. The shape and size of the handle (60) may be determined by a design choice and environments and conditions of the user.

In certain embodiments of the invention, the urinal device (100) may further comprise a stand (70), on which the urinal device (100) in the above.

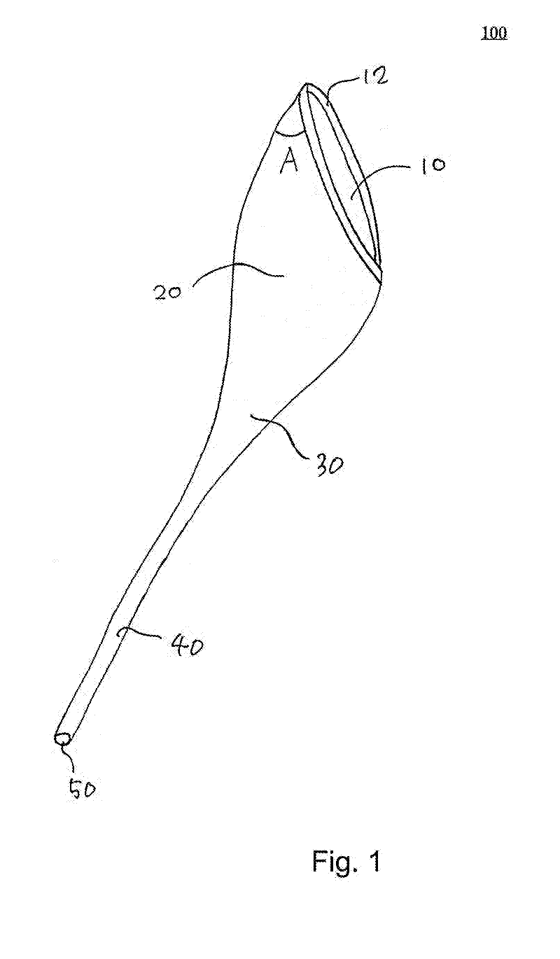

Referring FIG. 1, the urinal device (100) may further comprise a rubber pad (12). In certain embodiments of the invention, the reservoir portion (20) may be made of flexible material, and the drain pipe portion (40) may be made of hard material. The upper opening (10) and the reservoir portion (20) may meet each other at an angle (A). And, the angle (A) may be 90 degrees or less than 90 degrees. That is, the angle (A) may be chosen according to design choices.

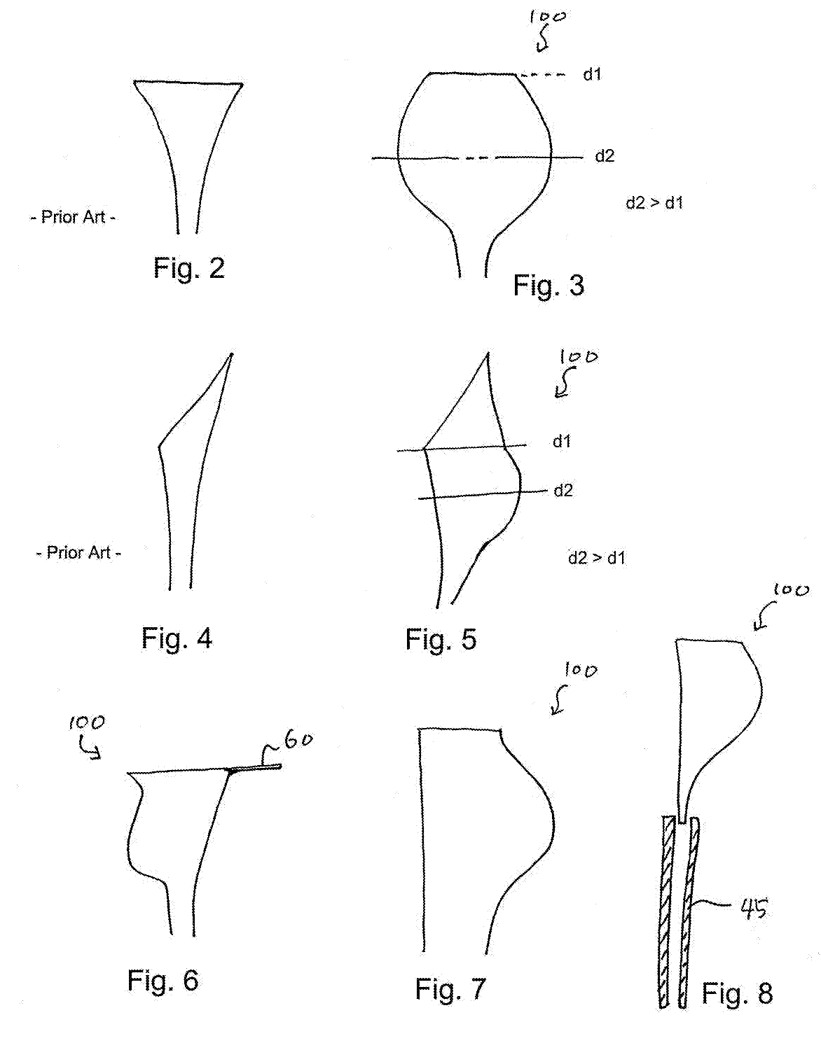

FIG. 3 can be compared to FIG. 2 (prior art). The dimension (d2) of the reservoir portion (20) must be larger than that (d1) of the upper opening (10) as shown in FIG. 3.

FIG. 5 can be compared to FIG. 4 (prior art). The dimension (d2) of the reservoir portion (20) must be larger than that (d1) of the upper opening (10) as shown in FIG. 5.

FIG. 6 shows a handle (60), which can have any shapes if satisfying the requirements of a general handle as shown in other figures.

As shown in FIG. 7, the bulge of the reservoir portion (20) may be one-sided. Or, it can bulge all around.

FIG. 8 shows a cross-sectional view of an extension pipe (45), which is configured to receive the drain pipe portion (40), extending the length thereof.

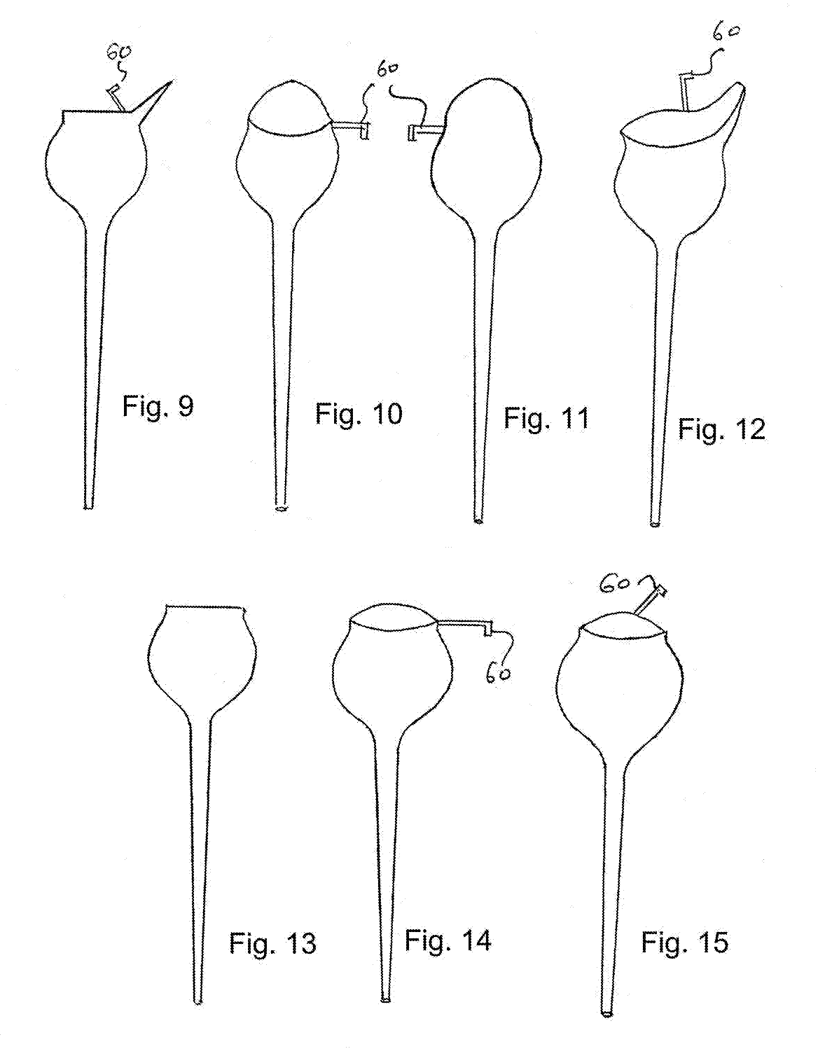

As shown in FIGS. 9-12, the rim of the upper opening (10) may be elevated or extended to other directions from the plane of the rest of the upper opening (10). Then, the reservoir portion (20) may accommodated to the change as illustrated.

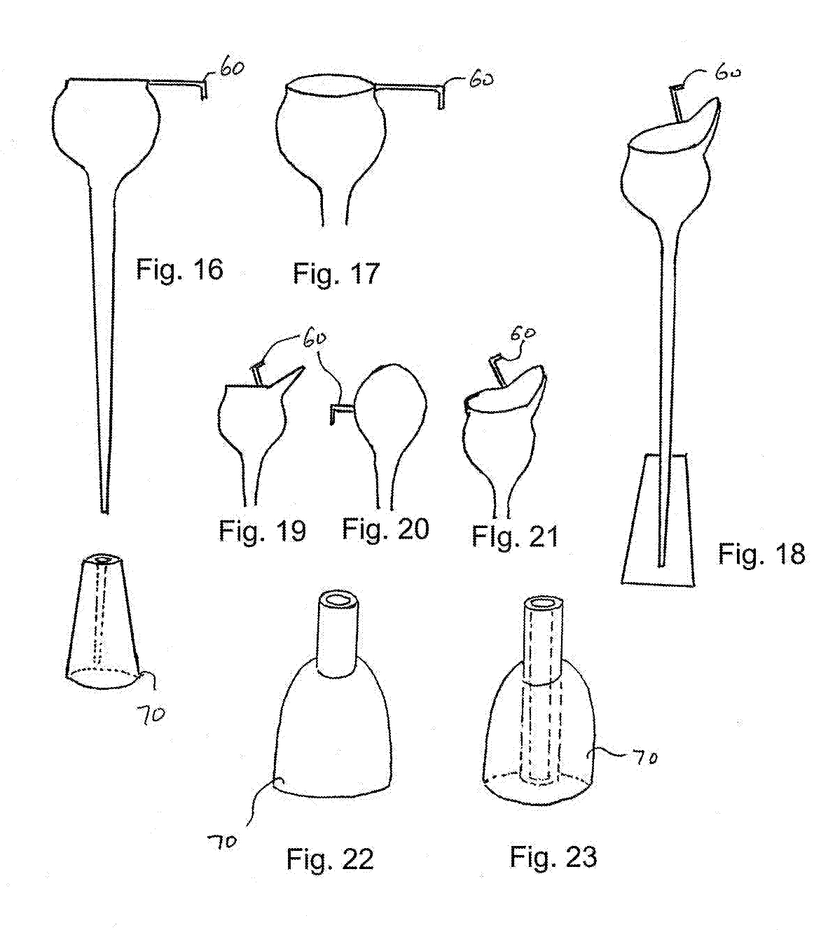

As shown in FIGS. 16-23, the urinal device (100) may further comprise a stand (70). It may help to store the upper part in place in the restroom.

The advantages of the present invention are: (1) the urinal device for men according to the invention is easy to make; and (2) the urinal device for men according to the invention is convenient to use.

An aspect of the invention provides a urinal device (100), comprising an upper opening (10), a reservoir portion (20), a funnel portion (30), a drain pipe portion (40), and a lower opening (50) as shown in FIG. 1. The upper opening (10) is configured for receiving urine from above.

The reservoir portion (20) extends downwards from and is formed below the upper opening (10) and configured for holding urine received through the upper opening (10) temporarily.

The funnel portion (30) extends downwards from and is formed below the reservoir portion (20) and configured for draining urine from the reservoir portion (20).

The drain pipe portion (40) extends downwards from and is formed below the funnel portion (30) and configured for draining urine from the funnel portion (30).

The lower opening (50) is formed at a lowest end of the drain pipe portion (40).

The reservoir portion (20) has a largest cross-sectional area between the upper opening (10) and the funnel portion (30), so that the reservoir portion (20) has a volume enough to hold urine.

A portion connecting from the upper opening (10) to the reservoir portion (20) may be tilted like a portion of a reversed funnel, as in a middle-bulged wine cup as shown in FIG. 3, for an example.

The urinal device (100) may further comprise a rubber pad (12) along the upper opening (10).

The urinal device (100) may further comprise a handle (60)extending from an edge of the upper opening (10) as shown in FIGS. 6, 9, 14, or the reservoir portion (20).

The upper opening (10) may be disposed flatly (refer to FIGS. 3, 7, 13) or obliquely with respect to a horizontal direction (refer to FIGS. 1, 5, 12, 21).

The reservoir portion (20) may be bulged to a single direction only as shown in FIGS. 1, 5-8.

The urinal device (100) according to the invention may further comprise a stand (70) having a stable bottom portion and a receiving hole provided on a top portion, which is configured to receive the lower part of the drain pipe portion (40) and the lower opening (50) as shown in FIGS. 16, 18, 22-23.

The stable bottom portion may be heavy enough to provide a center-of-mass low enough for the urinal device (100) to stand stably.

The top portion of the stand (70) may extend upwards from the stable bottom portion as shown in FIGS. 22-23.

The urinal device (100) may further comprise an extension pipe (45), to which the lower part of the drain pipe portion (40) close to the lower opening (50) is configured to be inserted. In such a case, the drain pipe portion (40) may be provided short as shown in FIG. 8.

The extension pipe (45) may be detachable.

The reservoir portion (20) may be made of flexible material.

The urinal device (100) may be made of plastic integrally or monolithically.

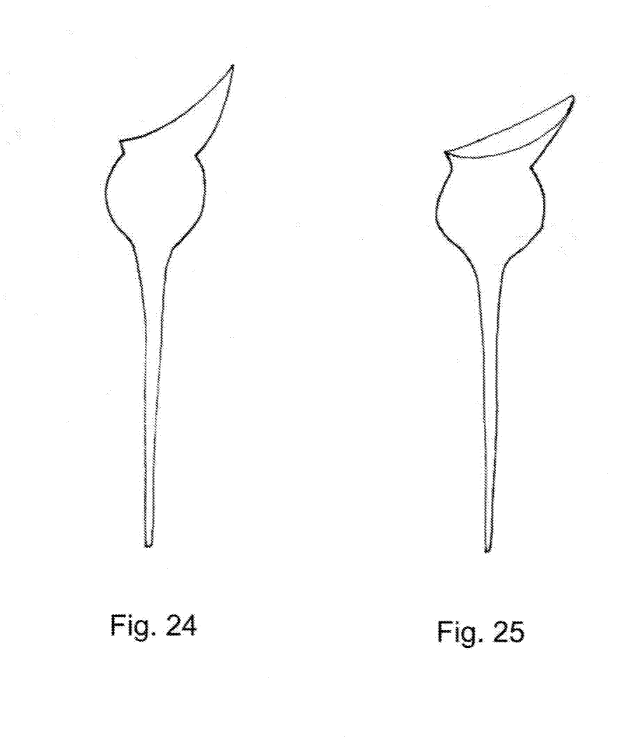

FIGS. 24 and 25 show different views of a urinal device according to still another embodiment of the invention. The shape of the opening may be varied according to situations.

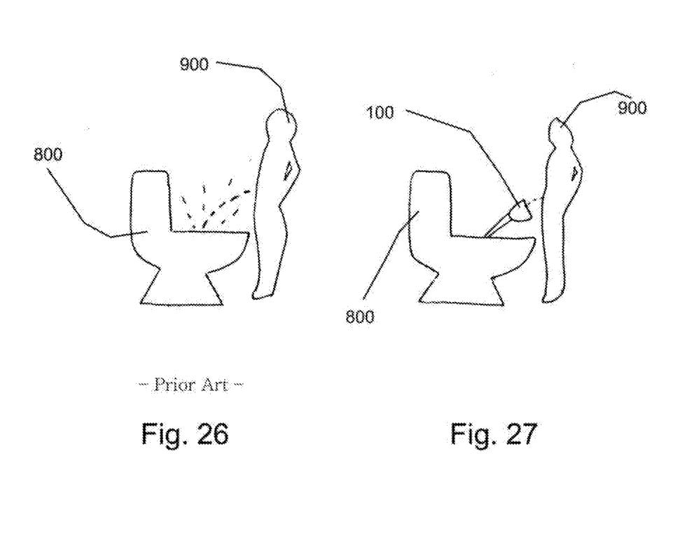

FIG. 26 shows a user (900) using a urinal (800) according to a prior art. The splash off from the inside of the conventional situation.

In contrast, in FIG. 27, a user (800) pees into the urinal device (100) according to embodiments of the invention, and therefore, there is no splash from the urinal (800).

The effects of the invention can be seen clearly by comparing the two situations in FIGS. 26 and 27.

While the invention has been shown and described with reference to different embodiments thereof, it will be appreciated by those skilled in the art that variations in form, detail, compositions and operation may be made without departing from the spirit and scope of the invention.

What is claimed is:

1. A urinal device comprising:
an upper opening configured for receiving urine from above;
a reservoir portion extending downwards from and formed below the upper opening and configured for holding urine received through the upper opening temporarily;
a funnel portion extending downwards from and formed below the reservoir portion and configured for draining urine from the reservoir portion;
a drain pipe portion extending downwards from and formed below the funnel portion and configured for draining urine from the funnel portion; and
a lower opening formed at a lowest end of the drain pipe portion,
wherein the reservoir portion has a largest cross-sectional area between the upper opening and the funnel portion, so that the reservoir portion has a volume enough to hold urine.

2. The urinal device of claim 1, wherein a portion connecting from the upper opening to the reservoir portion is tilted like a portion of a reversed funnel, as in a middle-bulged wine cup.

3. The urinal device of claim 1, further comprising a rubber pad along the upper opening.

4. The urinal device of claim 1, further comprising a handle extending from an edge of the upper opening or the reservoir portion.

5. The urinal device of claim 1, wherein the upper opening is disposed flatly or obliquely with respect to a horizontal direction.

6. The urinal device of claim 1, wherein the reservoir portion is bulged to a single direction only.

7. The urinal device of claim 1, further comprising a stand having a stable bottom portion and a receiving hole provided on a top portion, which is configured to receive the lower part of the drain pipe portion and the lower opening.

8. The urinal device of claim 7, wherein the stable bottom portion is heavy enough to provide a center-of-mass low enough for the urinal device to stand stably.

9. The urinal device of claim 7, wherein the top portion of the stand extends upwards from the stable bottom portion.

10. The urinal device of claim 1, further comprising an extension pipe, to which the lower part of the drain pipe portion close to the lower opening is configured to be inserted.

11. The urinal device of claim 10, wherein the extension pipe is detachable.

12. The urinal device of claim 1, wherein the reservoir portion is made of flexible material.

13. The urinal device of claim 2, wherein the urinal device is made of plastic integrally.
